(19) United States
(12) Patent Application Publication
(10) Pub. No.: US 2019/0358078 A1
(43) Pub. Date: Nov. 28, 2019

(54) NASAL DEVICE

(71) Applicant: SHU-CHEN TSAI, Taipei City (TW)

(72) Inventor: Yoshiaki Akihiro, Taipei City (TW)

(21) Appl. No.: 16/028,602

(22) Filed: Jul. 6, 2018

(30) Foreign Application Priority Data
May 22, 2018 (TW) 107117402

(51) Int. Cl. A61F 5/56

(52) U.S. Cl. CPC A61F 5/56

(57) ABSTRACT

This invention provides a nasal device for use while breathing and adapted for insertion within a human nasal cavity, the device comprising: a main body being tubular in shape having a hollow structure which extends from the end of front side to the end of rear side; a front opening portion and a rear opening portion; wherein the hollow structure is in a shape that the rear side becomes narrower towards the front side having a smaller diameter. The nasal device in this invention is easy to carry, which can be placed inside nasal cavity to bring sufficient air into the respiratory tract.

### FIELD OF THE INVENTION

This invention provides a nasal device to be placed inside the nasal cavity while breathing.

### BACKGROUND OF THE INVENTION

Sleep apnea syndrome, characterized by frequent pauses in breathing during sleep, is a common occurrence in the general population. When symptoms of sleep apnea occur, reduction-oxidation reaction in blood is insufficient, causing oxygen deficiency when blood circulates to brain. Therefore, it causes bad influences on not only the cranial nerve tissue, but also the hormone metabolism. Moreover, it may increase risk of lifestyle disease such as heart attack, diabetes or cerebral vascular disease.

The traditional method of inserting a nasal expander into nasal cavity during sleep to relieve the symptoms of sleep apnea syndrome is disclosed in Japan Patent Number 2006-501889. This type of nasal expander is placed inside nasal cavity while expanded to improve air flow into nostrils. However, for those who have severe sleep apnea syndrome, it is difficult to lead air into respiratory tract and this type of device is very uncomfortable to the user.

Comparing to the traditional method of treating sleep apnea syndrome patients, the positive airway pressure treatment is also disclosed in Japan Patent Number 2011-189170. The treatment requires that patients wear a face mask attached to a device for supplying pressurized air, and the device sends a continuous flow of pressurized air into patients' respiratory tract to maintain airway patency during sleep. This positive airway pressure treatment comparing to the aforementioned nasal expander can indeed sends airflow into the patient's respiratory tract. However, the treatment is required every night and is very uncomfortable for the patient. When the patient is away from home, the patient will need to bring both the pressurized air device and the face mask, which is inconvenient.

### SUMMARY OF THE INVENTION

This invention aims to provide a nasal device, which is easy to carry and can be placed within the nasal cavity to lead sufficient airflow into respiratory tract.

A nasal device for use while breathing and adapted for insertion within a human nasal cavity, the device comprising: a main body being tubular in shape having a hollow structure which extends from the end of front side to the end of rear side, wherein the main body is insertable into a nasal cavity; a front opening portion having an opening of said hollow structure in the end of front side of main body and opens towards the outside of the device; and a rear opening portion having an opening of said hollow structure in the end of rear side of main body and opens towards the outside of the device; wherein the hollow structure is in a shape that the rear side becomes narrower towards the front side having a smaller diameter.

While breathing with the main body of nasal device inserted in the nasal cavity, air flows into the hollow structure from rear opening portion, the airflow speeds up when passing through the rear side end and the front side end, and exits through the front opening portion and enters into the respiratory tract.

### BRIEF DESCRIPTION OF THE DRAWINGS

FIG. 1 is a perspective view of the nasal device in accordance with a first embodiment of the present invention.

FIG. 2 is a perspective view of the nasal device in accordance with a first embodiment of the present invention.

FIG. 3 is a top view of the nasal device in accordance with a first embodiment of the present invention.

FIG. 4 is a cross-sectional view taken along line A-A of FIG. 3.

FIG. 5 is an enlarged cross-sectional view of the main body of the nasal device in accordance with a first embodiment of the present invention.

FIG. 6 is an enlarged cross-sectional view of the front side of the nasal device in accordance with a first embodiment of the present invention.

FIG. 7 is a representative drawing of the operation of the nasal device in accordance with a first embodiment of the present invention.

FIG. 8 is a front view of the nasal device in accordance with a second embodiment of the present invention.

FIG. 9 is a cross-sectional view of the main body of the nasal device in accordance with a second invention of the present invention.

FIG. 10 is a cross-sectional view of the attachment of the nasal device in accordance with the second embodiment of the present invention.

FIG. 11 is a representative drawing of the nasal device in accordance with a third embodiment of the present invention.

### DETAILED DESCRIPTION OF THE INVENTION

The embodiments below are used for illustrating the objectives and effects of the present invention, and are not to be construed as constituting any limitations of the invention.

### Embodiment 1

### [Configuration of Nasal Device]

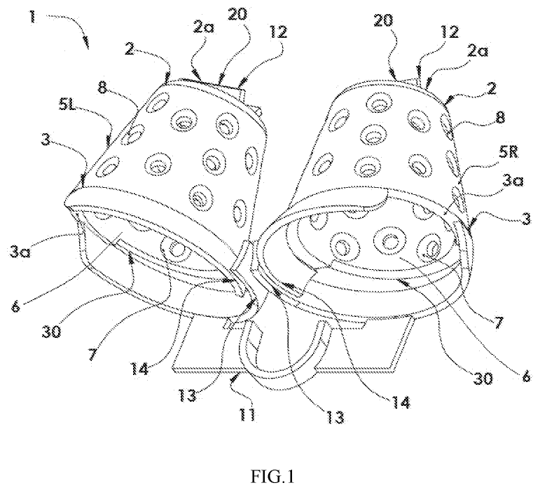

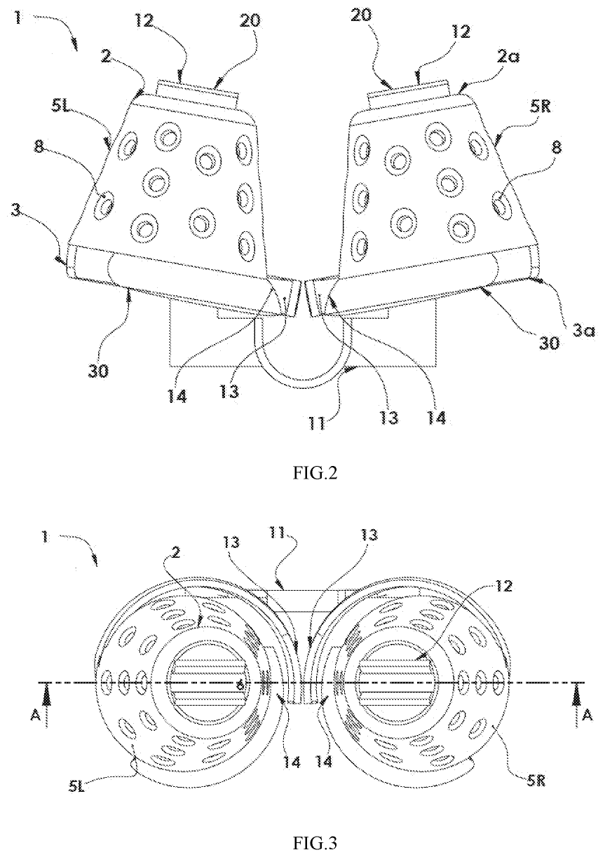

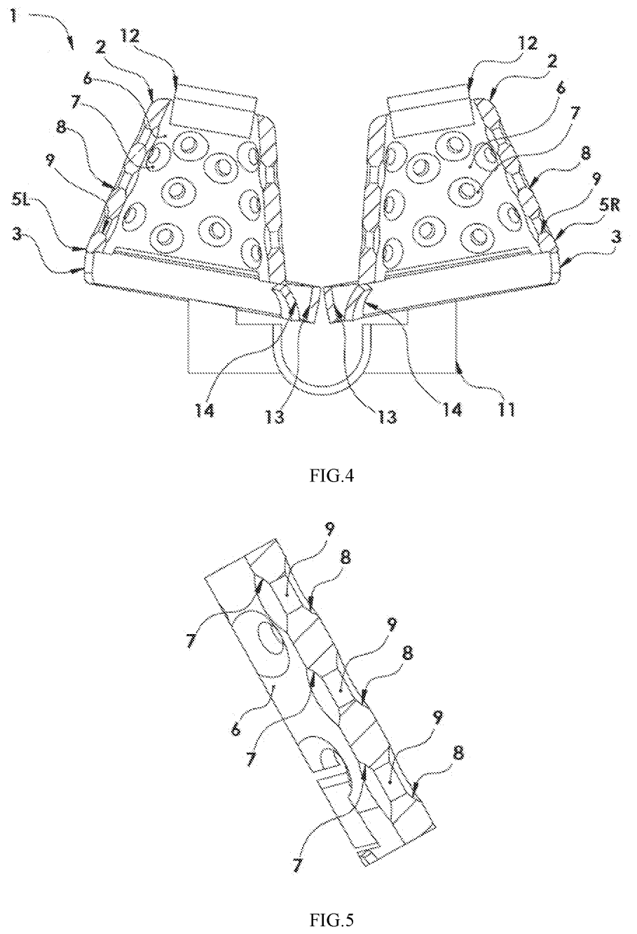

Referring to FIGS. 1 to 6, the nasal device 1 in embodiment 1 of the present invention is described below.

The nasal device 1 is integrally formed from elastic synthetic resin and the entire device exhibits elastic deformation,. Examples of synthetic resin in nasal device 1 that may be used include, but are not limited to: styrene elastomer, polyester elastomer, silicon, polyethylene and polyvinyl chloride.

The nasal device 1 comprises a left main body 5L that is insertable into the left nasal cavity, a right main body 5R that is insertable into the right nasal cavity, and a connector 11 that connects the left main body 5L and the right main body 5R. Both the left main body 5L and right main body 5R are symmetric in appearance.

The left main body 5L and the right main body 5R are both tubular in shape, and the end 3a of rear side 3 of each main body becomes narrower towards the end 2a of front side 2 having a smaller diameter, which results in a cone-shaped main body. The main body is insertable into the nasal cavity from the front side 2. More specifically, the left main body 5L and the right main body 5R each comprises a hollow structure 6, an internal concave part 7, an external concave part 8, a pathway 9, a front opening portion 20, a rear opening portion 30, guiding plates 12, a pressing portion 13 and a stopper 14.

The hollow structure 6 located inside the left main body 5L and right main body 5R extends from the end 2a of front side 2 to the end 3a of rear side 3, and the rear side 3 becomes narrower towards the front side 2 having a smaller diameter.

The front opening portion 20 having an opening of hollow structure 6 in the end 2a of front side 2 opens the hollow structure 6 towards the outside of the device.

The rear opening portion 30 having an opening of the hollow structure 6 in the end 3a of rear side 3 opens the hollow structure 6 towards the outside of the device. The inner diameter of rear opening portion 30 is larger than that of the front opening portion 20.

The pressing portion 13 is a part of the rear opening portion 30 which extends circularly outwards from the junctions of connector 11 that connects the left main body 5L and the right main body 5R from the rear side 3. The pressing portion 13 is capable of elastically deforming in an inward direction. When the left main body 5L and the right main body 5R is inserted into the user's nasal cavity, the pressing portion 13 is placed at the corresponding position of musculus depressor septi nasi of the left main body 5L and the right main body 5R to apply pressure to the musculus depressor septi nasi. The pressing portion 13 expands circularly outwards and inclines outwards, which is similar to the rear side 3 of the hollow structure 6 that expands circularly outwards.

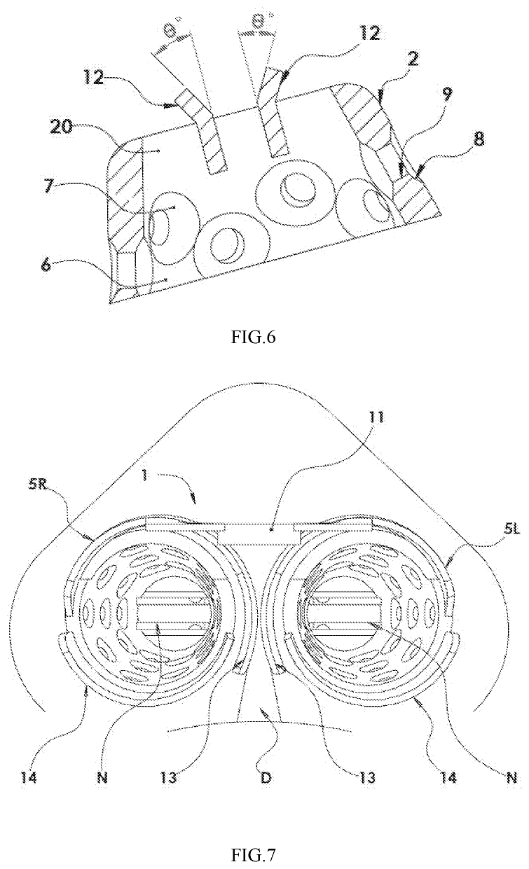

Guiding plates 12 are in tabular shape and work in a pair. Guiding plates 12 are located at the inner surface of the hollow structure 6 at the front side 2, and they extend outwardly towards the outside of the device in a direction away from each other. A pair of guiding plates 12 in FIG. 6 is inclined at a certain angle of inclination against the axis of the hollow structure 6. In this embodiment, the suitable angle would be 0° to 30°.

The internal concave part 7 in FIG.5 is a bowl-shaped concave part formed by gouging the inner surface of the left main body 5L and the right main body 5R surrounding the hollow structure 6, wherein a plurality of internal concave parts are located at the inner surface.

The external concave part 8 in FIG.5 is a bowl-shaped concave part formed by gouging the exterior surface of the left main body 5L and the right main body 5R, wherein a plurality of concave parts are located at the opposite side of the internal concave part 7.

The pathway 9 in FIG.5 connects the hollow structure 6 to the outside of the device, wherein one end is the opening of internal concave part 7 and the other end is the opening of exterior concave part 8.

The stopper 14 is a part of the rear opening portion 30, which moves accordingly to the pressure portion 13 when the pressure portion 13 elastically deforms. The stopper 14 expands circularly in a similar manner to the rear side 3 of the hollow structure 6 that inclines outwards.

The left main body 5L and the right main body 5R with said structure are positioned on the basis of extending the axis of the hollow structure 6 of the left main body 5L and the axis of the hollow structure 6 of the right main body 5R, where the axis meet in front of the left main body 5L and the right main body 5R.

### [Operation of Nasal Device]

Referring to FIGS.1 to 7, the operation of the nasal device 1 in this invention is described in details below.

As shown in FIG. 1, elasticity of the connector 11 of the nasal device 1 keeps the left main body 5L and the right main body 5R almost parallel. The left main body 5L is then inserted into the left nasal cavity N from the front side 2 and the right main body 5R is inserted into the right nasal cavity N from the front side 2 simultaneously. When the nasal device 1 is inserted within the nasal cavity, the left main body 5L and right main body 5R gently exert a force on the inner wall of nasal cavity N and elastically deform inwards. At the same time, the resilience of connector 11 causes the left main body 5L and right main body 5R to rebound back to the original form depicted in FIG. 1. The form where the left main body 5L and right main body 5R gently press the inner wall of nasal cavity N can prevent the nasal device from falling out of the nasal cavity N.

When breathing with the nasal device 1 inserted in nasal cavity N, outside air flows from the rear opening portion 30 into the hollow structure 6, and the airflow speeds up from the rear side 3 of the hollow structure 6 to the front side 2, then exits through the front opening portion 20 of nasal cavity N and enters into the respiratory tract. Accordingly, this nasal device can be placed in nasal cavity N and transport sufficient air into the respiratory tract.

The expanding diameter of rear side 3 can minimize the resistance of airflow being flowed through the rear opening portion 30 to the hollow structure 6, and minimize the reduction of air speed when air flows from the rear opening portion 30 to the hollow structure 6.

Moreover, air flows through the hollow structure 6 and turbulent flow is created by the spiral-shaped internal concave part 7, which accelerates the flow rate of air in hollow structure 6.

Additionally, the movement of air in internal concave part 7 causes air in external concave part 8 and pathway 9 to be sucked into internal concave part 7. Comparing to the internal concave part 7, external concave part 8 and pathway 9 form a negative pressure, thus allowing the mucus secreted from nose to evaporate to external concave part 8 and pathway 9 easily. The mucus evaporated to external concave part 8 and pathway 9 is drawn into the internal concave part 7 and reaches the hollow structure 6, and then enters into the deep parts of nasal cavity N or mucosa tissue of the pharynx by airflow in the hollow structure 6. Therefore, the deep parts of nasal cavity N or mucosa tissue of the pharynx may remain moist and protect their functions.

Furthermore, the flow rate of air exhaled from the nasal cavity N through the front opening portion 20 can be accelerated by guiding plates 12, and guiding plates 12 can guide the airflow to of the middle meatus of nose and exhaled out, without reducing the speed when going into the larynx. It has been experimentally confirmed that by having the guiding plates 12 inclined 30 degrees with the axis of hollow structure 6, the flow rate of air exhaled from the nasal cavity N through the front opening portion 20 can be accelerated.

During use, the nasal device 1 may be used to accelerate the air speed when air flows into the nasal cavity N, or inhibit the reduction of air flow rate when air flows into the nasal cavity N, thereby accelerating the air flow rate to reach lungs and causing the pulmonary alveoli to further inflate. Moreover, by further inflating the pulmonary alveoli, its elastic recoil is more effective in deflating the pulmonary alveoli and accelerating the flow rate of exhaled air. It can also reduce the turbidity caused by the pause between inhalation and exhalation while air passes through bronchi, thereby increasing the respiratory capacity and increase peripheral capillary oxygen saturation (SpO2).

FIG. 7 depicts the form of nasal device 1 when it is inserted within nasal cavity N. The pressing portion 13 is elastically deformed inwards, as the inner wall of nasal cavity N presses the pressing portion 13. Meanwhile, the resilience of pressing portion 13 releases the energy absorbed and applies a force to the musculus depressor septi nasi D. The volume of nasal cavity is enlarged downwards and thereby more air from the outside may be inhaled into the nasal cavity N and exhaled more air out of the nasal cavity N.

Additionally, when the stopper 14 is not placed, there is a possibility that the pressing portion 13 may be greatly elastically deformed inwards, causing chances of injuring nasal cavity mucosa while the end of rear side is clamped onto the mucosa. Therefore, while pressing portion 13 presses musculus depressor septi nasi D and elastically deforms, by placing the stopper 14 at the corresponding location of moving direction of pressing portion 13 thereby preventing the nasal cavity N from being injured.

Additionally, the pressing portion 13 presses the inner wall of nasal cavity N in an outward direction and allows the surrounding musculature of nasal cavity N to expand.

According to this embodiment, the shape of hollow structure 6, wherein the rear side becomes narrower towards the front side having a smaller diameter, thereby allowing sufficient air to enter the respiratory tract without the pressurized air device and face mask.

According to this embodiment, the connector 11 connects the left main body 5L and the right main body 5R to form the nasal device 1 for allowing sufficient air to enter into the respiratory tract without the pressurized air device and face mask. The nasal device 1 is easily portable.

According to this embodiment, the connector 11 connects the left main body 5L and the right main body 5R and prevents any of the left main body 5L or right main body 5R from being lost or forgotten to bring by a user.

Additionally, in this embodiment, although the left main body 5L and right main body 5R are in cone-shaped, the left main body 5L and right main body 5R may be cylindrical in shape from the rear side to front side and as long as the hollow structure 6 comprises a rear side 3 that becomes narrower towards the front side 2.

### Embodiment 2

### [Configuration of the Nasal Device]

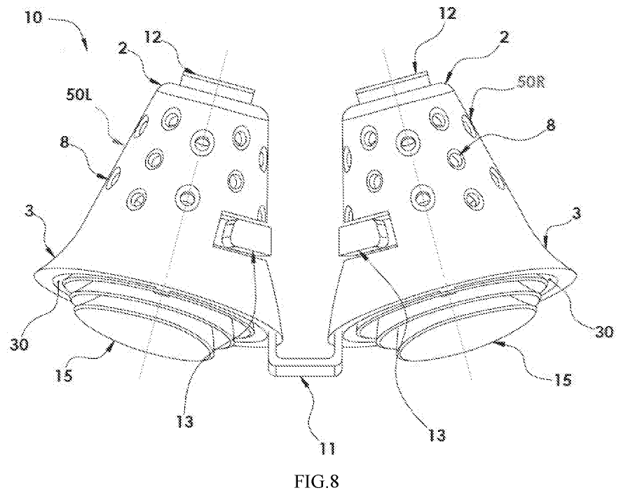

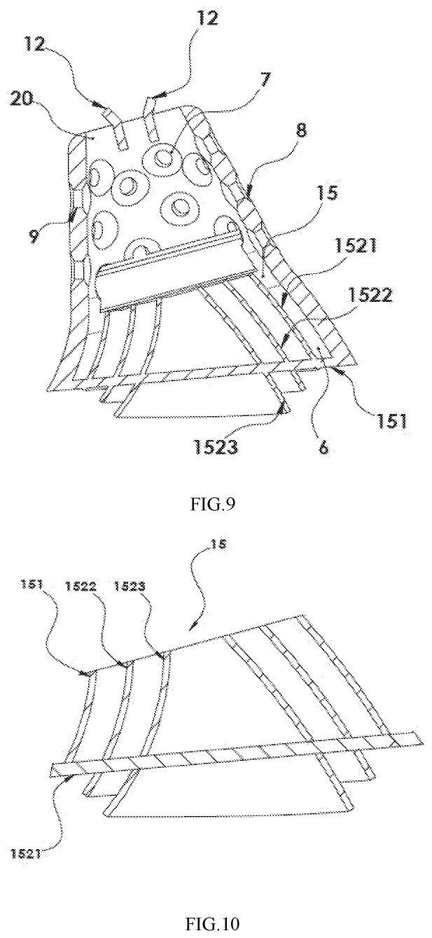

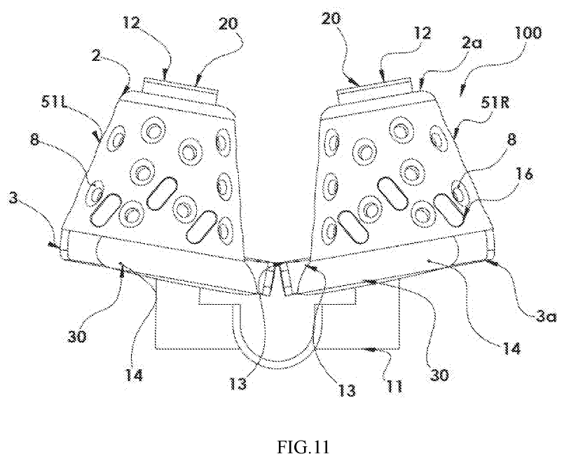

Referring to FIGS.8-10, the nasal device 10 of embodiment 2 in this invention is described in details below.

In FIGS.8-10, the components that are identical to those in FIGS.1-7 are marked with the same reference numbers and letters, and their descriptions are omitted.

The nasal device 10 is integrally formed from elastic synthetic resin and the entire device exhibits elastic deformation. Examples of synthetic resin in nasal device 10 that may be used include, but are not limited to: styrene elastomer, polyester elastomer, silicon, polyethylene and polyvinyl chloride.

The nasal device 10 comprises a left main body 50L that is insertable into the left nasal cavity, a right main body 50R that is insertable into the right nasal cavity, and a connector 11 that connects the left main body 50L and the right main body 50R. Both the left main body 50L and right main body 50R are symmetric in appearance.

The left main body 50L and the right main body 50R are both tubular in shape, and the end 3a of rear side 3 of each main body becomes narrower towards the end 2a of front side 2 having a smaller diameter, which results in a cone-shaped main body. The main body is insertable into the nasal cavity from the front side 2, wherein the rear side 3 extending upwardly towards the rear end. The rear side 3 of the left main body 50L and the rear side 3 of the right main body 50R gradually extend towards the rear end and form into cone-shaped structures. More specifically, the left main body 50L and the right main body 50R each comprises a hollow structure 6, an internal concave part 7, an external concave part 8, a pathway 9, a front opening portion 20, a rear opening portion 30, guiding plates 12, a pressing portion 13, a stopper 14 and an attachment 15.

The attachment 15 which is located at the rear side 3 of the left main body 50L and the right main body 50R is removable from the left main body 50L and the right main body 50R. The attachment 15 is conical in shape and gradually increases its diameter towards the rear end, and gradually extends in an upward direction. The attachment 15 is a partition structure comprising a locking component 151, a partition 1521, a partition 1522 and a partition 1523.

The locking component 151 is rod-shaped and connects a partition 1521, a partition 1522, a partition 1523, and said component is integrally formed. Both ends of long side of the locking component are removable and lockable on the inner surface.

The partition 1521 placed between the inner surface and the partition 1522 maintains a desired distance between the inner surface and the partition 1522 and is constructed along the inner surface and the partition 1522. The partition 1521 divides the hollow structure 6 along the diameter of the hollow structure 6. The partition 1521 is located deeper than the rear opening portion 30. The partition 1521 gradually increases its diameter towards the rear end and gradually extends in an upward direction, which results in a cone-shaped body.

The partition 1522 placed between the partition 1521 and the partition 1523 maintains a desired distance between the partition 1521 and the partition 1523 and is constructed along the partition 1521 and the partition 1523. The partition 1522 divides the hollow structure 6 along the diameter of the hollow structure 6. The partition 1522 extends outwards from the rear opening portion 30 towards the outside of the device. The partition 1522 gradually increases its diameter towards the rear end and gradually extends in an upward direction, which results in a cone-shaped body.

The partition 1523 placed at the inner side of the partition 1522 maintains a desired distance with the partition 1522 and is constructed along the partition 1522. The partition 1523 divides the hollow structure 6 along the diameter of the hollow structure 6. The partition 1523 extends more outwards than the partition 1522 from the rear opening portion 30 towards the outside of the device. The partition 1523 gradually increases its diameter towards the rear end and gradually extends in an upward direction, which results in a cone-shaped body. The internal space of partition 1523 gradually broadens towards the rear end.

The rear side 3 of the hollow structure 6 comprises structures of said partition 1521, a partition 1522, a partition 1523, and divides the hollow structure 6 into a plurality of spaces along the diameter of the hollow structure.

### [Operation of Nasal Device]

Referring to FIGS.8-10, operation of the nasal device 10 of embodiment 2 in this invention is described in details below.

The left main body 50L is inserted into the front side 2 of the left nasal cavity, and the right main body 50R is inserted into the front side 2 of the right nasal cavity simultaneously.

When breathing with the nasal device 10 inserted in nasal cavity N, outside air flows from the rear opening portion 30 into the hollow structure 6, and the airflow speeds up from the rear side 3 of the hollow structure 6 to the front side 2, then exits through the front opening portion 20 of nasal cavity N. Accordingly, this nasal device can transport sufficient air from the nasal cavity into the respiratory tract.

The expanding diameter of the rear side 3 of the main left body 50L and the right main body 50R in cone-shaped can minimize the resistance of airflow being flowed through the rear opening portion 30 to the hollow structure 6, and minimize the reduction of air speed when air flows from the rear opening portion 30 to the hollow structure 6.

Additionally, since the space between the inner surface and the partition 1521, the space between the partition 1521 and the partition 1522, the space between the partition 1522 and the partition 1523, and the space surrounded by the partition 1523 are all separated from each other, the flow rate of air in this space can be accelerated by the attachment 15 comparing to situations without placing the attachment 15.

Additionally, with the rear side 3 of the left main body 50L, the rear side 3 of the right main body 50R and the attachment 15, the device extends towards the rear end with an increasing diameter and becomes cone-shaped. Hence, it can minimize the resistance of airflow being flowed from outside to the left main body 50L, the right main body 50R and the attachment 15, and minimize the reduction of air speed when air flows from outside to the left main body 50L, the right main body 50R and the attachment 15.

Additionally, the rear side 3 of the left main body 50L, the rear side 3 of the right main body 50R and the attachment 15 gradually extends in an upward direction towards the rear end, thereby when inserting the nasal device 50 into the nasal cavity, a front side opening is formed by the rear end of left main body 50L, the rear end of the right main body 50R, and the attachment 15. Consequently, outside air can easily flow into the left main body 50L, the right main body 50R and the attachment 15.

Moreover, the partition 1522 and the partition 1523 extend in an outward direction from the rear opening portion 30 towards the outside of the device, thereby allowing air farther away to be flowed from the attachment 15 into the hollow structure 6.

Moreover, the operation to accelerate the air flow rate of nasal device 10 is the same as accelerating air flow rate of the nasal device 1 in another embodiment, hence it is omitted in embodiment 2.

According to this embodiment, the shape of hollow structure 6 wherein the rear side becomes narrower towards the front side having a smaller diameter, thereby allowing sufficient air to enter the respiratory tract without the pressurized air device and face mask.

According to this embodiment, the connector 11 connects the left main body 50L and the right main body 50R to form the nasal device 10 for allowing sufficient air to enter into the respiratory tract without the device that supplies air and face mask. The nasal device 10 is easily portable.

According to this embodiment, the attachment 15 at the rear side 3 divides the hollow structure 6 into a plurality of spaces along the diameter of the hollow structure 6 and accelerates the air inflow from outside to the rear opening portion 30 and into the rear side 3 of the hollow structure 6. For patients who cannot breathe in sufficient air by themselves, the attachment 15 allows sufficient air to enter the nasal cavity and into the respiratory tract without the pressurized air device and face mask.

According to this embodiment, the left main body 50L and the right main body 50R comprises of the attachment 15 which is removable from said left main body and right main body, can help patients who use the nasal device 10 to adjust and breathe in suitable air capacity based on the symptoms they are having.

Additionally, in this embodiment, although the left main body 50L and right main body 50R are in cone-shaped, the left main body 50L and right main body 50R may be cylindrical in shape from the rear side 3 to the front side 2 and as long as the hollow structure 6 comprises a rear side 3 that becomes narrower towards the front side 2.

The left main body 50L and the right main body 50R with said structure are positioned on the basis of extending the axis of the hollow structure 6 of the left main body 50L and the axis of the hollow structure 6 of the right main body 50R, where the axis meet in front of the left main body 50L and the right main body 50R.

Additionally, the attachment 15 in this embodiment comprises three partitions: a partition 1521, a partition 1522 and a partition 1523. However, the attachment may also comprise one, two or four and above partitions.

Additionally, the attachment 15 in this embodiment is removable from the left main body 50L and the right main body 50R. However, the attachment may also be unremovable and integrally formed with the left main body 50L and the right main body 50R.

Additionally, the partition 1522 and the partition 1523 in this embodiment extend in an outward direction from the rear opening portion 30 towards the outside of the device. However, one or more of the partition 1521, the partition 1522 and the partition 1523 may extend in an outward direction from the rear opening portion 30 towards the outside of the device. Alternatively, all of the partition 1521, the partition 1522 and the partition 1523 may extend in an outward direction from the rear opening portion 30 towards the outside of the device, or none of said partitions may extend in an outward direction from the rear opening portion 30 towards the outside of the device.

### Embodiment 3

This embodiment is an adaptation of the first embodiment and the second embodiment. Referring to FIG.11, this embodiment uses the adaptation of embodiment 1 as an example, which is described in details below.

In FIG.11, the components that are identical in those in FIGS.1-7 are marked with the same reference numbers and letters, and their descriptions are omitted.

The nasal device 100 is integrally formed from elastic synthetic resin and the entire device exhibits elastic deformation. Examples of synthetic resin in nasal device 100 that may be used include, but are not limited to: styrene elastomer, polyester elastomer, silicon, polyethylene and polyvinyl chloride.

The nasal device 100 comprises a left main body 51L that is insertable into the left nasal cavity, a right main body 51R that is insertable into the right nasal cavity, and a connector 11 that connects the left main body 51L and the right main body 51R. Both the left main body 51L and right main body 51R are symmetric in appearance.

The left main body 51L and the right main body 51R are both tubular in shape, and the end 3a of each main body becomes narrower towards the end 2a having a smaller diameter, which results in a cone-shaped body. Each of the main body comprises a plurality of diagonal slits and the said main body is insertable into the nasal cavity from the front side 2. More specifically, the left main body 51L and the right main body 51R each comprises a hollow structure 6, an internal concave part 7, an external concave part 8, a pathway 9, a front opening portion 20, a rear opening portion 30, guiding plates 12, a pressing portion 13, a stopper 14 and diagonal slits 16.

When the nasal device 100 is inserted in the nasal cavity, the nasal cavity opening (vestibulum nasi) where the device is inserted has certain width. Although the size of vestibulum nasi varies little, it still differs from one person to another. When the nasal device 100 is in use, it is best that the main body 51 is adhered closely to the nasal mucosa.

The left main body 51L and the right main body 51R of the nasal device comprise a plurality of diagonal slits 16, and therefore when inserting the nasal device, the pressure forces onto the left main body 51L and the right main body 51R is evenly applied to the diagonal slits 16, and the left main body 51L and the right main body 51R will shrink and become narrower. Thus, the left main body 51L and the right main body 51R do not harm or cause pain when being placed inside the nasal cavity. The nasal device is easy to insert and after said device it is in place, the pressure released and the elastic restoring force (resilience) of the left main body 51L and the right main body 51R triggers said main bodies to rebound to their original form, thereby causing the left main body 51L and the right main body 51R to adhere closely to the nasal cavity mucosa.

Referring to said diagonal slits 16, the inclination angle, quantity, and distribution can be designed accordingly.

Moreover, the operation of nasal device 100 is the same as the nasal device 1, hence it is omitted. Breathing with the nasal device 100 inside the nasal cavity can also allow sufficient air to enter the respiratory tract without the pressurized air device and face mask.

Moreover, a plurality of diagonal slits 16 can also be positioned on the nasal device 10 in the second embodiment in order to fit the main body closely to the nasal cavity when using the nasal device 10.

The types, configuration and quantity of the components in this invention are not limited to the aforementioned embodiments. Proper modifications to change the components and achieve the same results are possible within the scope of the invention.

More specifically, in the said embodiments 1-3, the left main body and the right main body connected by the connector can also be separated and used without a connector.

Although the invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### INDUSTRIAL APPLICABILITY

The present invention is carried out in the nasal device which is used in the nasal cavity while breathing.

What is claimed is:

1. A nasal device for use while breathing and adapted for insertion within a human nasal cavity, the device comprising: a main body being tubular in shape having a hollow structure which extends from the end of front side to the end of rear side, wherein the main body is insertable into a nasal cavity; a front opening portion having an opening of said hollow structure in the end of front side of main body and opens said hollow structure towards the outside of the device; and a rear opening portion having an opening of said hollow structure in the end of rear side of main body and opens said hollow structure towards the outside of the device; wherein the hollow structure is in a shape that the rear side becomes narrower towards the front side having a smaller diameter.

2. The nasal device of claim 1, wherein the main body comprises a plurality of internal concave parts on the inner surface of said main body surrounding said hollow structure.

3. The nasal device of claim 2, wherein the main body comprises a plurality of external concave parts on the exterior surface of said main body surrounding said hollow structure, and a plurality of pathways from the opening of internal concave parts to the opening of external concave parts at the opposite side, which connect said hollow structure to the outside of the device.

4. The nasal device of claim 1, wherein the main body comprises a pressing portion which applies a force to the musculus depressor septi nasi of a human nasal cavity when the device is inserted within the nasal cavity.

5. The nasal device of claim 1, wherein the main body comprises a plurality of guiding plates, which are oppositely placed at the front side and extend outwardly towards the outside of device.

6. The nasal device of claim 1, wherein the main body comprises a partition structure, which is placed at the rear side and divides said hollow structure into a plurality of spaces along the diameter of hollow structure.

7. The nasal device of claim 6, wherein the partition structure extends outwardly from the rear opening portion towards the outside of the device.

8. The nasal device of claim 6, wherein the partition structure is removable from said main body.

9. The nasal device of claim 1, wherein the main body includes a plurality of diagonal slits.
